(19) United States
(12) Patent Application Publication
(10) Pub. No.: US 2007/0092515 A1
(43) Pub. Date: Apr. 26, 2007

(54) Methods of treating chronic pain using compositions that specifically bind cd11d (alpha-d) integrin

(75) Inventors: Lynne C. Weaver, London (CA); Gregory Dekaban, London (CA); Mark Oatway, Mississauga (CA)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Icos Corporation, Bothell, WA (US)

(21) Appl. No.: 10/578,562

(22) PCT Filed: Nov. 8, 2004
(86) PCT No.: PCT/US04/37245

§ 371 Date: Dec. 13, 2006

Related U.S. Application Data
(60) Provisional application No. 60/517,843, filed on Nov. 6, 2003.

(51) Int. Cl. A61K 39/395

(52) U.S. Cl. 424/144.1

(57) ABSTRACT

Methods to treat secondary injury resulting from spinal cord injury using polypeptide compositions that specifically bind to CD11d are disclosed.

### FIELD OF THE INVENTION

The present invention relates to methods for treating or preventing chronic pain using compositions that specifically bind integrin subunit alpha-d (α_{d}), also known as CD11d. The present invention provides anti-CD11d-specific polypeptide compositions that may be administered in conjunction with existing pain therapy to synergistically prevent or alleviate symptoms of chronic pain in an individual.

### BACKGROUND OF THE INVENTION

The integrins are a class of membrane-associated molecules which actively participate in cellular adhesion. Integrins are transmembrane heterodimers comprising an α subunit in noncovalent association with a β subunit. To date, at least eighteen α subunits and eight β subunits have been identified [reviewed in van der Flier et al., Cell Tissue Res. 305:285-98 (2001); Takagi et al., Immunol Rev. 186:141-63 (2002); and Springer, Nature 346:425-434 (1990)]. The β subunits are generally capable of association with more than one α subunit and the heterodimers sharing a common β subunit have been classified as subfamilies within the integrin population.

One class of human integrins, restricted to expression in white blood cells, is characterized by a common β2 subunit. As a result of this cell-specific expression, these integrins are commonly referred to as the leukocyte integrins, Leu-CAMs or leukointegrins. An alternative designation of this class is the β2 integrins. The β2 subunit (CD18) has previously been isolated in association with one of four distinct α subunits, CD11a, CD11b, CD11c or CD11d. The isolation of a cDNA encoding human CD18 is described in Kishimoto et al., Cell 48:681-690 (1987). In official WHO nomenclature, the heterodimeric proteins are referred to as CD11a/CD18, CD11b/CD18, CD11c/CD18, and CD 11d/CD18; in common nomenclature they are referred to as LFA-1, Mac-1 or Mo1, p 150,95 or LeuM5, and α_{d}β₂, respectively [Cobbold, et al. in Leukocyte Typing III, McMichael (ed), Oxford Press, p. 788 (1987); Van der Vieren et al., Immunity 3:683-690 (1995)]. DNAs encoding the human β2 integrin α subunits CD11a, CD11b, CD11c and CD11d have been cloned [CD11a, Larson et al., J. Cell Biol. 108:703-712 (1989); CD11b, Corbi et al., J. Biol. Chem. 263:12403-12411 (1988); CD11c, Corbi et al. EMBO J. 6:4023-4028 (1987); CD11d, Van der Vieren et al., Immunity 3:683-690 (1995)]. Putative homologs of the human β2 integrin α and β chains, defined by approximate similarity in molecular weight, have been variously identified in other species including monkeys and other primates [Letvin et al., Blood 61:408-410 (1983)], mice [Sanchez-Madrid et al., J. Exp. Med. 154:1517 (1981)], and dogs [Moore et al., Tissue Antigens 36:211-220 (1990); Danilenko et al., J. Immunol. 155:35-44 (1995)].

In humans, CD11a/CD18 is expressed on all leukocytes. CD11b/CD18 and CD11c/CD18 are essentially restricted to expression on monocytes, granulocytes, macrophages and natural killer (NK) cells, but CD11c/CD18 is also detected on some B-cell types. In general, CD11a/CD18 predominates on lymphocytes, CD11b/CD18 on granulocytes and CD11c/CD18 on macrophages [see review, Arnaout, Blood 75:1037-1050 (1990)]. CD11d is expressed primarily on neutrophils and monocytes/macrophages, but also found on natural killer cells and subsets of B and T cells [Grayson et al., J. Exp. Med. 188:2187-91 (1998)]. Expression of the α chains, however, is variable with regard to the state of activation and differentiation of the individual cell types [See review, Larson and Springer, Immunol. Rev. 114:181-217 (1990)]

The involvement of the β2 integrins in human immune and inflammatory responses has been demonstrated using monoclonal antibodies which are capable of blocking β2 integrin-associated cell adhesion. For example, CD11a/CD18, CD11b/CD18 and CD11c/CD18 actively participate in natural killer (NK) cell binding to lymphoma and adenocarcinoma cells [Patarroyo et al., Immunol. Rev. 114:67-108 (1990)], granulocyte accumulation [Nourshargh et al., J. Immunol. 142:3193-3198 (1989)], granulocyte-independent plasma leakage [Arfors et al., Blood 69:338-340 (1987)], chemotactic response of stimulated leukocytes [Arfors et al., supra] and leukocyte adhesion to vascular endothelium [Price, et al., J. Immunol. 139:4174-4177 (1987) and Smith et al., J. Clin. Invest. 83:2008-2017 (1989)].

Interestingly, at least one antibody specific for CD18 has been shown to inhibit human immunodeficiency virus type-1 (HIV-1) syncytia formation in vitro, albeit the exact mechanism of this inhibition is unclear [Hildreth et al., Science 244:1075-1078 (1989)]. This observation is consistent with the discovery that a principal counterreceptor of CD11a/CD18, ICAM-1, is also a surface receptor for the major group of rhinovirus serotypes [Greve et al., Cell 56:839 (1989)].

Adhesion and extravasation of leukocytes are mediated by integrins. The CD11/CD18 integrins on the surface of leukocytes bind to adhesion molecules such as intercellular adhesion molecules (ICAMs) and vascular cell adhesion molecule-1 (VCAM-1) on endothelia [Bevilacqua et al., Ann. Rev. Immunol. 11:7670-804 (1993). These integrins appear to play an important role in leukocyte trafficking and activation of phagocytic activity, and mediate cell-cell interactions during inflammation [Petty et al., Immunol. Res. 25:75-95 (2002); Miranti et al., Nat. Cell Biol. 4:E83-E90 (2002); Schwartz et al., Nat. Cell Biol. 4:E65-E68 (2002)]. The CD11d subunit binds to VCAM-1 in rats and ICAM-3 and VCAM-1 in humans [Grayson et al. (1998); Van der Vieren et al. (1999); Van der Vieren, et al. (1995)]

Acute therapies after spinal cord injury have yet to offer a practical way to control the early inflammatory response leading to chronic pain. Drugs such as methylprednisolone (MP) have failed to yield satisfactory clinical results, perhaps because the modest neuroprotective effects [Bracken, Spine 26:547-854 (2001)] are outweighed by harmful side effects [Hurlbert, Spine 26:539-546 (2001)]. At the subacute phase of injury, artificially increasing “active immunity” by delivering activated macrophages or by vaccination that directs T-lymphocytes to the injured spinal [Hauben et al., Lancet 355:286-287 (2000); Schwartz et al., J. Neuroimmunol. 113:185-192 (2001)] has clinical potential, but may lead to autoimmune disorders [Jones et al., J. Neurosci. 22:2690-2700 (2002)].

Thus, there exists a need in the art for a method for treatment of pain which reduces harmful side effects and accurately targets chronic pain disorders affecting all animals, such as humans.

### SUMMARY OF THE INVENTION

The present invention provides improved treatment for secondary injury and chronic pain resulting from trauma to the spinal cord. The present invention provides methods for treating chronic pain using antibodies and polypeptide compositions that bind to the alpha-d integrin subunit, i.e., CD11d.

In one aspect, the invention provides methods for treating chronic pain in a mammalian subject comprising the step of administering to a subject in need a therapeutically effective amount of a composition comprising a polypeptide that specifically binds CD11d. In one embodiment, the composition of the methods comprises an antibody. In another embodiment, methods are modified wherein the composition comprises a monoclonal antibody. In a related embodiment, the composition of the methods comprises a monoclonal antibody secreted by hybridoma 217L (deposited Apr. 30, 1999 with the American Type Culture Collection, Manassas, Va. 20110, as Accession No: HB-12701), hybridoma 226H or hybridoma 236L (both deposited Nov. 11, 1998 with American Type Culture Collection as Accession No: HB-3-12592 and Accession No: HB-12593, respectively).

The invention contemplates methods wherein the polypeptide composition that specifically binds CD11d comprises a polypeptide comprising one, two and/or three complementarity determining regions (CDR) of a light chain of a monoclonal antibody secreted by hybridoma 217L, 226H or 236L. It is further contemplated that the composition of the methods comprises a polypeptide comprising one, two and/or three complementarity determining region (CDR) of a heavy chain of monoclonal antibody secreted by hybridoma 217L, 226H or 236L. The invention further provides that the composition of the methods comprises a polypeptide comprising one, two and/or three complementarity determining regions (CDR) of a heavy chain of monoclonal antibody secreted by hybridoma 217L, 226H or 236L, and one, two and/or three complementarity determining regions (CDR) of a light chain of monoclonal antibody secreted by hybridoma 217L, 226H or 236L.

The methods of the invention provide for treatment of chronic pain wherein the administered composition comprises a polypeptide that recognizes an epitope on CD11d recognized by a monoclonal antibody secreted by hybridoma 217L, 226H or 236L. It is further contemplated that the composition of the methods comprises a polypeptide that competes with a monoclonal antibody secreted by hybridoma 217L, 226H or 236L for binding to CD11 d.

The invention provides methods wherein the composition comprises a polypeptide comprising one, two, three, four, five and/or six complementarity determining regions of a monoclonal antibody secreted by hybridoma 217L, 226H or 236L, said polypeptide selected from the group consisting of a monoclonal antibody, a polyclonal antibody, a single chain antibody, a chimeric antibody, a bifunctional/bispecific antibody, a humanized antibody, a human antibody, and a complementarity determining region (CDR)-grafted antibody and a peptibody.

The mammalian subject treated by the methods of the invention may be human, or any non-human animal model for human medical research, or an animal of importance as livestock or pets, for example, companion animals. In a preferred variation, the subject has a disease or condition characterized by a need for amelioration or elimination of the symptoms of chronic pain, and administration of a composition comprising a polypeptide that specifically binds CD11d and that results in improvements in the animal's state, for example by palliating disease symptoms, slowing chronic pain progression, curing the chronic pain, or otherwise improving clinical symptoms. In a preferred embodiment, the subject to be treated is human.

The methods of the invention provide treatment for chronic pain. The invention contemplates that the chronic pain is selected from the group consisting of tactile allodynia, neuropathic pain, hyperalgesia, hyperpathia, and inflammatory pain. In a preferred embodiment, the chronic pain being treated is tactile allodynia.

Additionally contemplated is the treatment of causalgia, post-operative pain, chronic lower back pain, cluster headaches, postherpetic neuralgia, phantom limb and stump pain, central pain, dental pain, neuropathic pain, opioid-resistant pain, visceral pain, surgical pain, bone injury pain, diabetic neuropathy pain, post-surgery or traumatic neuropathy pain, peripheral neuropathy pain, entrapment neuropathy pain, neuropathy caused by alcohol abuse, pain from HIV infection, multiple sclerosis hypothyroidism or anticancer chemotherapy pain, pain during labor and delivery, pain resulting from burns, including sunburn, postpartum pain, migraine, angina pain, and genitourinary tract-related pain including cystitis.

In one aspect of the invention, the chronic pain results from central nervous system trauma or injury to the spinal cord. It is contemplated that the trauma or injury may be a result of secondary injury arising from inflammation in the central nervous system, as would result from microbial (e.g., bacterial, fungal, viral) infection, cancerous cells in the CNS, or other edema or encephalopathy. It is further contemplated that inflammation may result from the effects of a CNS-related autoimmune disease, such as multiple sclerosis.

Additionally contemplated methods of the invention are the treatment of injuries occurring in the central nervous system, including physical injury, which causes the degeneration of the axonal processes and/or nerve cell bodies near the site of the injury; ischemia, such as from a stroke; exposure to neurotoxins, such as cancer and AIDS chemotherapeutic agents; chronic metabolic diseases, such as diabetes or renal dysfunction; and neurodegenerative diseases such as Parkinson's disease, Alzheimer's disease, and amyotrophic lateral sclerosis (ALS), which cause the degeneration of specific neuronal populations. Conditions involving nerve damage include Parkinson's disease, Alzheimer's disease, amyotrophic lateral sclerosis, stroke, diabetic polyneuropathy, toxic neuropathy, glial scar, and physical damage to the nervous system such as that caused by physical injury of the brain and spinal cord, such as compression injuries, or crush or cut/laceration injuries to the spinal cord, arm, hand or other parts of the body, including temporary or permanent cessation of blood flow to parts of the nervous system, as in stroke.

The methods of the invention provide treatment with compositions that reduce and ameliorate symptoms of chronic pain in the subject being treated. In one aspect, the treatment with compositions described herein results in an increase in appropriate axon growth and/or regeneration. In another aspect, the treatment with compositions described herein results in an increase in myelination. An improvement in myelin density and appropriate axon growth and/or regeneration is measured by techniques well-known in the art, including magnetic resonance imaging (MRI), biopsy of patient tissue sample, and responses to neurological examination. In a further aspect, the treatment may reduce injury- or inflammation-induced abnormal growth of axons that leads to chronic pain. In a related aspect, improvement of chronic pain, as assessed by, for example, the Leeds Assessment of Neuropathic Symptoms and Signs (LANSS) Pain Scale, indicates an increase in axon and/or myelin growth and/or regeneration.

The invention further provides methods wherein the composition comprising a polypeptide that specifically binds CD11d is in a pharmaceutically acceptable diluent or carrier. In a related aspect, the composition is administered in conjunction with other pain relief medicines. In one aspect, the other pain relief medicine is selected from the group consisting of non-steroidal anti-inflammatory drugs (NSAIDs), analgesics, steroids, and anti-epileptic medicines.

In addition to the foregoing, the invention includes, as an additional aspect, all embodiments of the invention narrower in scope in any way than the variations specifically mentioned above. Although the applicants invented the fall scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicants reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE INVENTION

Methods of the invention for the treatment for chronic pain address the selective suppression of the early, destructive phase of inflammation in the central nervous system that results from an injured spinal cord. Suppression of the inflammatory response provides an opportunity for later regenerative interventions and wound-healing responses.

In order that the invention may be more completely understood, several definitions are set forth.

As used herein, the term “pain” shall refer to all types of pain. In one aspect, the term shall refer to acute and chronic pains, such as causalgia, tactile allodynia, neuropathic pain, hyperalgesia, hyperpathia, inflammatory pain, post-operative pain, chronic lower back pain, cluster headaches, postherpetic neuralgia, phantom limb and stump pain, central pain, dental pain, neuropathic pain, opioid-resistant pain, visceral pain, surgical pain, bone injury pain, diabetic neuropathy pain, post-surgery or traumatic neuropathy pain, peripheral neuropathy pain, entrapment neuropathy pain, neuropathy caused by alcohol abuse, pain from HIV infection, multiple sclerosis hypothyroidism or anticancer chemotherapy pain, pain during labor and delivery, pain resulting from burns, including sunburn, postpartum pain, migraine, angina pain, and genitourinary tract-related pain including cystitis.

By “therapeutically effective amount” is meant an amount of a composition comprising a polypeptide that specifically binds CD11d that, when administered alone, is effective in providing pain relief, at least partially or completely. “Concurrent administration,” “administered in combination” or similar phrases mean that the composition comprising a polypeptide that specifically binds CD11d and a pain relief medicine are administered concurrently to the mammal being treated. By “concurrently,” it is meant that each component may be administered at the same time or sequentially in any order at different points in time. However, if not administered at the same time, they should be administered sufficiently closely in time so as to provide the desired potentiation of treatment effect. Suitable dosing intervals and dosing order with such compounds will be readily apparent to those skilled in the art. It is also contemplated that a pain relief medication or other second agent, such as steroids, may also be administered prior to administration of a polypeptide that specifically binds CD11d. Prior administration refers to administration of the pain relief medication or second agent within the range of one week prior to anti-CD11d antibody/polypeptide treatment up to 30 minutes before administration of anti-CD11d. It is further contemplated that the second agent is administered subsequent to administration of a polypeptide that specifically binds CD11d. Subsequent administration is meant to describe administration from 30 minutes after administration of polypeptide that specifically binds CD11d up to one week after anti-CD11d antibody/polypeptide treatment “Polypeptide” or “anti-CD11d antibody/polypeptide” as used herein refers to polypeptides that specifically bind to and recognize the CD11d molecule. Polypeptides contemplated by the invention include monoclonal and polyclonal antibodies, single chain antibodies, chimeric antibodies, bifunctional/bispecific antibodies, humanized antibodies, human antibodies, Fab, Fab′, F(ab′)₂, Fv, single-chain antibodies, complementarity determining region (CDR)-grafted antibodies, including compounds that include one or more CDR sequences specifically recognizing the CD11d integrin, and peptibodies.

That a polypeptide “specifically binds” the CD11d molecule or is “CD11d-specific” or is “specific for” CD11d refers to the ability of a binding agent to recognize and bind CD11d, but not other integrins (or other antigens). In one aspect, the CD11d-binding polypeptides of the invention, or fragments, variants, or derivatives thereof, will bind with a greater affinity to human CD11d as compared to its binding affinity to CD11d of other, i.e., non-human, species, but binding polypeptides that recognize and bind orthologs are within the scope of the invention.

For example, a polypeptide that is an antibody “specific for” its cognate antigen indicates that the variable regions of the antibodies recognize and bind the polypeptide of interest with a detectable preference (i.e., able to distinguish the polypeptide of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody for use in the methods of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies for use in the invention can be produced using any method known in the art.

The term “antigen binding domain” or “antigen binding region” refers to the portion of the selective binding agent that contains the amino acid residues (or other moieties) that interact with an antigen and confer on the binding agent its specificity and affinity for the antigen.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three dimensional structure that is identical to the epitope used to generate the peptibody, yet comprise none or only some of the amino acid residues found in CD11d that were used to stimulate the peptibody immune response. As used herein, a mimotope is not considered a different antigen from the epitope bound by the selective binding agent; the selective binding agent recognizes the same three-dimensional structure of the epitope and mimotope.

The term “variable region” or “variable domain” refers to a portion of the light and/or heavy chains of an antibody, typically including approximately the amino-terminal 120 to 130 amino acids in the heavy chain and about 100 to 110 amino acids in the light chain, which differ extensively in sequence among antibodies and which determine the binding and specificity of each particular antibody for its particular antigen. The variability in sequence is concentrated in the complementarity-determining regions (CDRs), while the more highly conserved regions in the variable domain are called framework regions (FR). The CDRs of the light and heavy chains contain within them the amino acids which are largely responsible for the direct interaction of the antibody with antigen.

The term “vehicle” refers to a molecule that prevents degradation and/or increases half-life, reduces toxicity, reduces immunogenicity, or increases biological activity of a polypeptide composition. Exemplary vehicles include an Fc domain (which is preferred) as well as a linear polymer (e.g., polyethylene glycol (PEG), polylysine, dextran, etc.); a branched-chain polymer (See, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid; a cholesterol group (such as a steroid); a carbohydrate or oligosaccharide; or any natural or synthetic protein, polypeptide or peptide that binds to a salvage receptor.

Additional definitions are found in the specification to assist in understanding of the relevant parts of the description.

Chronic Pain

The present invention provides methods for alleviating and treating symptoms that arise in a subject experiencing chronic pain.

The causes of pain can include inflammation, injury, disease, muscle spasm and the onset of a neuropathic event or syndrome. Ineffectively treated pain can be detrimental to the person experiencing it by limiting function, reducing mobility, complicating sleep, and interfering with general quality of life.

Inflammatory pain can occur when tissue is damaged, as can result from surgery or due to an adverse physical, chemical or thermal event or to infection by a biologic agent. Although inflammatory pain is generally reversible and subsides when the injured tissue has been repaired or the pain inducing stimulus removed, present methods for treating inflammatory pain have many drawbacks and deficiencies.

Neuropathic pain is a persistent or chronic pain syndrome that can result from damage to the nervous system, the peripheral nerves, the dorsal root ganglion or dorsal root, or to the central nervous system. Neuropathic pain syndromes include, but are not limited to, allodynia or pain due to a typically non-noxious stimulus, various neuralgias such as post herpetic neuralgia and trigeminal neuralgia, phantom pain, and complex regional pain syndromes, such as reflex sympathetic dystrophy and causalgia. Allodynia involves a change in the quality of a sensation, whether tactile or mechanical, thermal, or of any other sort. A patient's original response to a stimulus may not involve pain per se, but the highly sensitized response indicates a loss of specificity of a sensory modality. Cutaneous allodynia is pain resulting from an innocuous stimulus to normal skin or scalp, and is believed to be caused by a transient increase in the responsiveness of central pain neurons that process information arising from the skin.

Neuralgia is defined as paroxysmal-pain that originates in a sensory nerve. Neuralgia is a local pain, usually severe, and felt in the area of the body from which a nerve normally carries sensation. However, it is pain caused by damage to the nerve itself, not by something being done to the part of the body that it serves.

Complex regional pain syndrome (CRPS), or Reflex Sympathetic Dystrophy, is a chronic condition characterized by severe burning pain, pathological changes in bone and skin, excessive sweating, tissue swelling, and extreme sensitivity to touch. One visible sign of CRPS near the site of injury is warm, shiny red skin that later becomes cool and bluish.

Causalgia is characterized by spontaneous burning pain combined with hyperalgesia and allodynia. Hyperalgesia is characterized by extreme sensitivity to a painful stimulus. (Meller et al., Neuropharmacol. 33:1471-8, 1994). This condition can include visceral hyperalgesia which generates the feeling of pain in internal organs. Neuropathic pain also includes hyperpathia, wherein a stimulus that is normally innocuous if given for a prolonged period of time results in severe pain.

Spasticity or muscle spasm can be a serious complication of trauma to the spinal cord or other disorders that create damage within the spinal cord and the muscle spasm is often accompanied by pain. The pain experienced during a muscle spasm can result from the direct effect of the muscle spasm stimulating mechanosensitive pain receptors or from the indirect effect of the spasm compressing blood vessels and causing ischemia. The spastic event increases the rate of metabolism in the affected muscle tissue. The relative ischemia becomes greater thereby creating conditions for the release of pain-inducing substances.

Chronic pain is difficult to treat since the anticipated side effects of pain relief medications, such as opioid or non-opioid analgesics, are difficult to manage. In the case of opioid analgesics, there is a high risk of addiction in patients experiencing chronic pain, as well as respiratory depression and constipation. For the non-opioid analgesics, such as non-steroidal anti-inflammatory drugs (NSAIDs), acetaminophen or aspirin, the risk of gastric ulcers may be dose limiting. Methods of the invention therefore improve treatment for chronic pain without the limiting side effects.

Non-Opioid Pain Relief Medications

In order to treat pain in a subject exhibiting a chronic pain disorder, the method of the invention may be administered in combination with other treatments, also referred to herein as “second agents,” used to relieve symptoms of chronic pain. These therapies include non-opioid medications such as NSAIDs, analgesics, and steroids, which have been shown to be therapeutic in neuropathies and are used to decrease inflammatory responses.

Exemplary NSAIDs contemplated for use in the invention are chosen from the group consisting of ibuprofen, naproxen, Cox-1 inhibitors, Cox-2 inhibitors, and salicylates. It is contemplated that all known or after discovered NSAIDs are useful for administration in the method of the invention in conjunction with compositions comprising a polypeptide that specifically binds CD11d.

Analgesics, used to treat chronic pain conditions, are contemplated for use in the method of the invention in combination with compositions comprising a polypeptide that specifically binds CD11d. It is contemplated that known or after discovered analgesics useful for treating chronic pain are also useful for administration in conjunction with compositions comprising a polypeptide that specifically binds CD11d.

Steroids, which have been shown to be therapeutic in neuropathies, and which in some instances non-specifically decrease inflammation, are contemplated for use in the methods of the invention in combination with compositions comprising a polypeptide that specifically binds CD11d. Steroids contemplated for use in the invention include, but are not limited to, androgens, estrogens, progestagens, 21-aminosteroids, glucocorticoids, steroid neurotransmitters (neuroactive steroids) and other steroid hormones known in the art. It, is contemplated that known or after discovered steroids, such as glucocorticoids, useful for treating chronic pain are useful for administration in conjunction with compositions comprising a polypeptide that specifically binds CD11d.

Polypeptide Compositions for Use in the Methods of the Invention

The nucleotide and amino acid sequence of the human CD11d integrin subunit are set out in SEQ ID NOs: 1 and 2. Any number of polypeptide compositions as described herein that specifically bind to the CD11d molecule are contemplated for use in methods of the invention.

For example, antibodies useful for detecting the CD11d protein or, fragments thereof are generated using techniques well known in the art. Thus, the invention contemplates use of antibodies (e.g., monoclonal and polyclonal antibodies, single chain antibodies, chimeric antibodies, bifunctional/bispecific antibodies, humanized antibodies, human antibodies, and complementarity determining region (CDR)-grafted antibodies, including compounds that include CDR sequences specifically recognizing the CD11d integrin for use in the method of the invention). Preferred antibodies are human antibodies that are produced and identified according to methods described in WO 93/11236, which is incorporated herein by reference in its entirety. Antibody fragments, including Fab, Fab′, F(ab′)₂, and Fv, and single-chain antibodies are also contemplated under the method of the invention.

Various procedures known in the art may be used for the production of polyclonal antibodies to peptides comprising CD11d. For the production of antibodies, various host animals (including but not limited to rabbits, mice, rats, hamsters, and the like) are immunized by injection with a CD11d protein or peptide (suitable immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG (Bacille Calmette-Guerin) and Corynebacterium parvum.

A monoclonal antibody to CD11d may be prepared by using any technique that provides for the production of antibody molecules by continuous cell lines in culture. These include but are not limited to the hybridoma technique originally described by Köhler et al., Nature, 256: 495-497 (1975), and the more recent human B-cell hybridoma technique [Kosbor et al., Immunology Today, 4:72 (1983)] and the EBV-hybridoma technique [Cole et al., Monoclonal Antibodies and Cancer Therapy, Alan R Liss, Inc., pp. 77-96 (1985), all specifically incorporated herein by reference]. Antibodies against CD11d also may be produced in bacteria from cloned immunoglobulin cDNAs. With the use of the recombinant phage antibody system it may be possible to quickly produce and select antibodies in bacterial cultures and to genetically manipulate their structure. Preparation of anti-CD11d monoclonal antibodies is exemplified in U.S. Pat. No. 6,432,404.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use hybridomas including, but not limited to, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use hybridomas including, but not limited to, R210.RCY3, Y3-Ag 1.2.3, IR983F and 4B210; and U-266, GM1500-GRG2, LICR-LON-HMy2 and UC729-6 in connection with cell fusions.

In addition to the production of monoclonal antibodies, techniques developed for the production of “chimeric antibodies,” the splicing of mouse antibody genes to human antibody genes to obtain a molecule with appropriate antigen specificity and biological activity, can be used [Morrison et al., Proc. Natl. Acad. Sci. 81:6851-6855 (1984); Neuberger et al., Nature 312:604-608 (1984); Takeda et al., Nature 314:452-454 (1985)]. Alternatively, techniques described for the production of single-chain antibodies (U.S. Pat. No. 4,946,778) can be adapted to produce CD11d-specific single chain antibodies.

Antibody fragments that contain the idiotype of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a CDR, of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into its framework region from a source which is non-human. Humanization can be performed, for example, using methods described in the art [e.g., Jones et al., Nature 321:522-525 (1986), Riechmann et al., Nature 332:323-327 (1988) and Verhoeyen et al., Science 239:1534-1536 (1988)], by substituting at least a portion of a rodent complementarity-determining region for the corresponding regions of a human antibody. Numerous techniques for preparing engineered antibodies are described the art [e.g., Owens et al., J. Immunol. Meth. 168:149-165, (1994)]. Further changes can then be introduced into the antibody framework to modulate affinity or immunogenicity.

Likewise, using techniques known in the art to isolate CDRs, compositions comprising CDRs are generated. CDRs are characterized by six polypeptide loops, three loops for each of the heavy or light chain variable regions. The amino acid position in a CDR is defined by Kabat et al., “Sequences of Proteins of Immunological Interest,” U.S. Department of Health and Human Services (1983), which is incorporated herein by reference. For example, hypervariable regions are roughly defined to be found at residues 28 to 35, from 49-59 and from residues 92-103 of the heavy and light chain variable regions [Janeway and Travers, Immunobiology, 2^{nd} Edition, Garland Publishing, New York (1996)]. It is understood in the art that CDR regions may be found within several amino acids of these approximated residues set forth above. An immunoglobulin variable region also consists of “framework” regions surrounding the CDRs. The sequences of the framework regions of different light or heavy chains are highly conserved within a species.

Polypeptide compositions comprising-one, two, and/or three CDRs of a heavy chain variable region or a light chain variable region of a monoclonal antibody are generated. For example, using the monoclonal antibody secreted by hybridoma 217L, polypeptide compositions comprising 217L-isolated CDRs are generated. Polypeptide compositions comprising one, two, three, four, five and/or six complementarity determining regions of a monoclonal antibody secreted by hybridoma 217L are also contemplated. Using the conserved framework sequences surrounding the CDRs, PCR primers complementary to these consensus sequences are generated to amplify the 217L CDR sequence located between the primer regions. Techniques for cloning and expressing nucleotide and polypeptide sequences are well-established in the art [see, e.g., Sambrook et al., Molecular Cloning: A Laboratory Manual, 2^{nd} Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

It is contemplated that modified polypeptide compositions comprising one, two three, four, five, and/or six CDRs of a monoclonal antibody of a heavy and/or light chain secreted by hybridoma 217L are generated, wherein a CDR is altered to provide increased specificity or affinity to the CD11d-molecule. Sites at locations in the 217L monoclonal antibody CDRs are typically modified in series, e.g., by substituting first with conservative choices (e.g., hydrophobic amino acid substituted for a non-identical hydrophobic amino acid) and then with more dissimilar choices (e.g., hydrophobic amino acid substituted for a charged amino acid), and then deletions or insertions may be made at the target site. It is contemplated that the CDR compositions discussed in relation to the 217L antibody may also be produced using CDRs from the 226H and 236L monoclonal antibodies or from other anti-CD11d antibodies.

“Conservative” amino acid substitutions are made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E). “Insertions” or “deletions” are preferably in the range of about 1 to 20 amino acids, more preferably 1 to 10 amino acids. The variation may be introduced by systematically making substitutions of amino acids in a polypeptide molecule using recombinant DNA techniques and assaying the resulting recombinant variants for activity. Nucleic acid alterations can be made at sites that differ in the nucleic acids from different species (variable positions) or in highly conserved regions (constant regions). Methods for expressing polypeptide compositions useful in the invention are described in greater detail below.

Rapid, large-scale recombinant methods for generating antibodies may be employed, such as phage display [Hoogenboom et al., J. Mol. Biol. 227: 381, (1991); Marks et al., J. Mol. Biol. 222: 581, (1991)] or ribosome display methods, optionally followed by affinity maturation [see, e.g., Ouwehand et al., Vox Sang 74 (Suppl 2):223-232 (1998); Rader et al., Proc. Natl. Acad. Sci. USA 95:8910-8915 (1998); Dall'Acqua et al., Curr. Opin. Struct. Biol. 8:443-450 (1998)]. Phage-display processes mimic immune selection through the display of antibody repertoires on the surface of filamentous bacteriophage, and subsequent selection of phage by their binding to an antigen of choice. One such technique is described in WO 99/10494, which describes the isolation of high affinity and functional agonistic antibodies for MPL and msk receptors using such an approach.

Bispecific antibodies are monoclonal, preferably human or humanized, antibodies that have binding specificities for at least two different antigens. Bispecific antibodies are produced, isolated, and tested using standard procedures that have been described in the literature. See, e.g., Pluckthun et al., Immunotechnology, 3:83-105 (1997); Carter et al., J Hematotherapy 4: 463-470 (1995); Renner & Pfreundschuh, Immunological Reviews 145:179-209 (1995); Pfreundschuh U.S. Pat. No. 5,643,759; Segal et al., J Hematotherapy 4:377-382 (1995); Segal et al., Immunobiology, 185:390-402 (1992); and Bolhuis et al., Cancer Immunol. Immunother. 34:1-8 (1991), all of which are incorporated herein by reference in their entireties.

The term “bispecific antibody” refers to a single, bivalent antibody that has two different antigen binding sites (variable regions). As described below, the bispecific binding agents are generally made of antibodies, antibody fragments, or analogs of antibodies containing at least one complementarity determining region derived from an antibody variable region. These may be conventional bispecific antibodies, which can be manufactured in a variety of ways [Holliger et al., Curr. Opin. Biotechnol. 4:446-449 (1993)], e.g., prepared chemically, using hybrid hybridomas, by placing the coding sequence of such a bispecific antibody into a vector and producing the recombinant peptide, or by phage display. The bispecific antibodies may also be any bispecific antibody fragments.

In one method, bispecific antibodies fragments are constructed by converting whole antibodies into (monospecific) F(ab′)₂ molecules by proteolysis, splitting these fragments into the Fab′ molecules and recombination of Fab′ molecules with different specificity to bispecific F(ab′)₂ molecules (see, for example, U.S. Pat. No. 5,798,229).

A bispecific antibody can be generated by enzymatic conversion of two different monoclonal antibodies, each comprising two identical L (light chain)-H (heavy chain) half molecules and linked by one or more disulfide bonds. Each monoclonal antibody is converted into two F(ab′)₂ molecules, splitting each F(ab′)₂ molecule under reducing conditions into the Fab′ thiols. One of the Fab′ molecules of each antibody is activated with a thiol activating agent and the active Fab′ molecule are combined, wherein an activated Fab′ molecule bearing one specificity is linked with a non-activated Fab′ molecule bearing an second specificity or vice versa in order to obtain the desired bispecific antibody F(ab′)₂ fragment.

Another method for producing bispecific antibodies is by the fusion of two hybridomas to form a hybrid hybridoma, as defined previously. Using now standard techniques, two antibody producing hybridomas are fused to give daughter cells, and those cells that have maintained the expression of both sets of clonotype immunoglobulin genes are then selected.

To identify the bispecific antibody, standard methods such as ELISA are used wherein the wells of microtiter plates are coated with a reagent that specifically interacts with one of the parent hybridoma antibodies and that lacks cross-reactivity with both antibodies. In addition, FACS, immunofluorescence staining, idiotype specific antibodies, antigen binding competition assays, and other methods common in the art of antibody characterization may be used in conjunction with the present invention to identify preferred hybrid hybridomas.

Also contemplated for use in the invention are peptibodies. Peptibody refers to a specific binding protein comprising at least one amino acid from a peptide, polypeptide or protein that interacts with CD11d alone, or fused to all or part of an immunoglobulin amino acid sequence, preferably a constant region, Fc, of an immunoglobulin, or other polypeptide carrier or vehicle described herein. Peptide fragments for use in making peptibodies may be from about 2 to 40 amino acids, with molecules of 3 to 20 amino acids preferred and those of 6 to 15 amino acids most preferred. Peptibodies are made by inserting an identified peptide sequence or peptide fragment that specifically binds to CD11d into a vector upstream or downstream of an Fc region, thereby creating a fusion product. The production of peptibodies is generally described in PCT publication WO 00/24782, incorporated herein by reference.

Any number of peptides that bind CD11d may be used in conjunction with the present invention. Phage display, in particular, is useful in generating peptides for use in the present invention as has been shown that affinity selection from libraries of random peptides can be used to identify peptide ligands for any site of any gene product [Dedman et al., J. Biol. Chien. 268:23025-30 (1993)].

Additional methods are employed to generate peptides for use in making peptibodies that specifically bind to CD11d. A peptide library can be fused to the carboxyl terminus of the lac repressor and expressed in E. coli. Another E. coli-based method allows display on the cell's outer membrane by fusion with a peptidoglycan-associated lipoprotein (PAL). These and related methods are collectively referred to as “E. coli display.” In another method, translation of random RNA is halted prior to ribosome release, resulting in a library of polypeptides with their associated RNA still attached. This and related methods are collectively referred to as “ribosome display.” Other methods employ chemical linkage of peptides to RNA. See, for example, Roberts and Szostak, Proc. Natl. Acad. Sci. USA 94:12297-303 (1997).

In the peptibody compositions prepared for use in the method of the invention, the peptide may be attached to an Fc domain or other polypeptide carrier or vehicle through the peptide's N-terminus or C-terminus or a side chain of one of the amino acid residues. Multiple vehicles may also be used; for example, an Fc domain at each terminus or an Fc at a terminus and a PEG group at the other terminus or a side chain. An Fc domain is the preferred vehicle. The Fc domain may be fused to the N or C termini of the peptides or at both the N and C termini.

Peptibody compositions also may comprise a “linker group.” The chemical structure of the linker is not critical, since it serves primarily as a spacer. The linker is preferably made up of amino acids linked together by peptide bonds. Thus, in one embodiment, the linker is made up of from 1 to 20 amino acids linked by peptide bonds, wherein the amino acids are selected from the 20 naturally occurring amino acids. One or more of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. Combinations of Gly and Ala are also preferred.

Non-peptide linkers are also possible. For example, alkyl linkers such as —NH—(CH₂)s-C(O)—, wherein s is a number from 2 to 20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker, and has a molecular weight of 100 to 5000 kDa, preferably 100 to 500 kDa.

Recombinant antibody fragments, for example, single chain Fv fragments, “scFv” fragments, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol. Eng. 18:95-108 (2001) and Todorovska et al., J Immunol Methods. 248:47-66 (2001).

Expression Vectors and Host Cells

Nucleotide sequences encoding the polypeptide compositions used in the invention are operably linked to an expression vector and transfected into an appropriate host cell for the expression of the polypeptide product. Suitable expression vectors and host cells are known in the art. Useful vectors include, e.g., plasmids, cosmids, viruses such as lambda phage and its derivatives, phagemids, artificial chromosomes, and the like, that are well known in the art. Mammalian expression vectors may comprise an origin of replication, a suitable promoter, and also any necessary ribosome binding sites, polyadenylation site, splice donor and/or acceptor sites, transcriptional termination sequences, and 5′ flanking nontranscribed sequences. DNA sequences derived from the SV40 viral genome, for example, the SV40 origin, early promoter, enhancer, splice, and polyadenylation sites may be used to provide the required expression control elements. Exemplary eukaryotic vectors include, without limitation, pcDNA3, pWVLneo, pSV2cat, pOG44, PXTI, pSG (Stratagene) pSVK3, pBPV, pMSG, and pSVL

Any host/vector system can be used to express one or more of the polynucleotides encoding polypeptides useful in the present invention. Proteins can be expressed in mammalian cells, yeast, bacteria, or other cells under the control of appropriate promoters. Appropriate cloning and expression vectors for use with prokaryotic and eukaryotic hosts are described by Sambrook et al., in Molecular Cloning: A Laboratory Manual, 2^{nd} Edition, Cold Spring Harbor, N.Y. (1989), the disclosure of which is hereby incorporated by reference. Mammalian host cells include, but are not limited to, monkey COS cells, Chinese Hamster Ovary (CHO) cells, human kidney HEK293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Also contemplated for use as host cells for expressing the chimeric polypeptide are insect cells, such as Sf9 cells. Any known viral expression system may also be used to generate the chimeric polypeptide, including, but not limited to, adenovirus, retrovirus, baculovirus [as described in Summers et al., Texas Agricultural Experiment Station Bulletin No. 1555 (1987)], and viral bacteriophages such as M13 or λ phage, being specifically contemplated.

Formulation of Pharmaceutical Compounds

It is contemplated that the composition comprising a polypeptide that specifically binds CD11d is administered to a subject in composition with one or more pharmaceutically acceptable carriers.

Pharmaceutical compositions comprising polypeptide compositions described herein are contemplated, and in one aspect the compounds are formulated with pharmaceutically acceptable diluents, adjuvants, excipients, and/or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal; retrobulbar, intrapulmonary injection and or surgical implantation at a particular site is contemplated as well. Generally, the compositions prepared are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions described above for use in the methods may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions useful in the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and/or coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions may also be in the form of suppositories for rectal administration of the antibody. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical forms suitable for injectable use include sterile aqueous solutions or dispersions and sterile powders for the extemporaneous preparation of sterile injectable solutions or dispersions. In all, cases the form must be sterile and must be fluid to the extent that easy syringability exists. It must also be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration and Dosing

The typical oral, parenteral or topical administration of an analgesic drug to treat the symptoms of pain can result in widespread systemic distribution of the drug and undesirable side effects. As such, the methods provide for improved methods of treating pain using compositions comprising a polypeptide that specifically binds to CD11d.

The invention contemplates that the compositions described herein are administered to a human or animal. Polypeptide compositions for use in the invention may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises a pharmaceutically-acceptable carrier solution such as water, saline, phosphate-buffered saline, glucose, or other carriers conventionally used to deliver therapeutics or imaging agents.

The “administering” that is performed according to the present invention may be performed using any medically-accepted means for introducing a therapeutic directly or indirectly into a mammalian subject, including but not limited to injections (e.g., intravenous, intramuscular, subcutaneous, intracranial or catheter); oral ingestion; intranasal or topical administration; and the like. In one embodiment, administering the composition is performed at the site of a lesion or affected tissue needing treatment by direct injection into the lesion site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations for use in the methods of the invention implanted near the lesion.

In another embodiment, the polypeptide composition is administered to a patient intrathecally. Intrathecal drug administration can avoid the inactivation of some drugs when taken orally as well the systemic effects of oral or intravenous administration. Additionally, intrathecal administration permits use of an effective dose which is only a fraction of the effective dose required by oral or parenteral administration. Furthermore, the intrathecal space is generally wide enough to accommodate a small catheter, thereby enabling chronic drug delivery systems.

It is possible to treat chronic pain and spasticity by intrathecal administration of a composition comprising a polypeptide that specifically binds CD11d. One current method for intrathecal treatment of chronic pain is by use of an intrathecal pump, such as the SYNCHROMED® Infusion System, a programmable, implanted pump available from Medtronic, Inc. (Minneapolis, Minn.). The pump is surgically placed under the skin of the patient's abdomen. One end of a catheter is connected to the pump, and the other end of the catheter is threaded into a CSF filled subarachnoid or intrathecal space in the patient's spinal cord. The implanted pump can be programmed for continuous or intermittent infusion of the polypeptide composition through the intrathecally located catheter.

The intraspinal administration of the compositions for use in the invention is carried out by intrathecal administration, such as intrathecally to a cranial, cervical, thoracic, lumbar, sacral or coccygeal region of the central nervous system and the administration step can include the steps of accessing a subarachnoid space of the central nervous system of the mammal, and injecting the CD11d polypeptide composition into the subarachnoid space. The accessing step can be carried out by spinal tap.

Alternately, the intraspinal administration step can include the steps of catheterization of a subarachnoid space of the central nervous system of the mammal, followed by injection of anti-CD11d mAb or other CD11d-specific polypeptide composition for use in the invention through a catheter inserted by the catheterization step into the subarachnoid space. Note that prior to the injecting step there can be the step of attaching to or implanting in the mammal an administration means for administering the CD11d-specific polypeptide composition to the central nervous system of the mammal.

It is important to note that the administration step can be carried out prior to the onset of or subsequent to the occurrence of a (inflammatory, neuropathic, injury induced, resulting form a cancer, spasm, etc.) event or syndrome experienced by the subject. In one aspect, the administration step can be carried out between 0.5 hour before to about 14 days before the onset of the injuring event. In another aspect, administering of the compositions after central nervous system trauma or injury may be carried out at any appropriate time, e.g., immediately after or up to 24 hours, 48 hours, or 72 hours after central nervous system trauma or injury. It is further contemplated that the polypeptide compositions may be administered for as long as the subject is in need of treatment, on a weekly, bi-weekly, or daily basis. Appropriate treatment regimens may be determined by the treating physician.

The therapeutic composition for use in the invention may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Polypeptides or antibodies for administration may be formulated with uptake or absorption enhancers to increase their efficacy. Such enhancers include for example, salicylate, glycocholate/linoleate, glycholate, aprotinin, bacitracin, SDS caprate and the like [see, e.g., Fix, J. Pharm. Sci. 85:1282-1285 (1996) and Oliyai and Stella, Ann. Rev. Pharmacol. Toxicol. 32:521-544 (1993)].

The amounts of pharmaceutical antibody composition in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day, 500 mg/day or 1000 mg/day. These doses may also be administered based on the patient's body weight, on a mg/kg/day basis. For example doses may be administered at 0.5 mg/kg/day, 1 mg/kg/day, 2 mg/kg/day, or as deemed appropriate by the treating physician. These concentrations may be administered as a single dosage form or as multiple doses. Standard dose-response studies, first in animal models and then in clinical testing, reveal optimal dosages for particular disease states and patient populations.

It will also be apparent that dosing should be modified if a traditional therapeutic is administered in combination with CD11d-specific polypeptide compositions used under the invention. For example, it is contemplated under the invention to treat tactile allodynia and related pain disorders using traditional analgesics, NSAIDs, or steroids, for example, estrogens, 21-aminosteroids or glucocorticoids, in combination with CD11d-specific polypeptide compositions.

Kits

As an additional aspect, the invention includes kits which comprise one or more compounds or compositions packaged in a manner which facilitates their use to practice methods of the invention. In a simplest embodiment, such a kit includes a compound or composition described herein as useful for practice of a method of the invention (e.g., a composition comprising a polypeptide that specifically binds CD11d), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay. Preferably, the kit contains a label that describes an approved use of the CD11d-specific polypeptide according to the invention, e.g., use of an anti-CD11 mAb for the treatment of chronic pain associated with central nervous system trauma, such as spinal cord injury.

Additional aspects and details of the invention will be apparent from the following examples, which are intended to be illustrative rather than limiting. Example 1 discloses that locomotor function after spinal cord injury was improved by the anti-CD11d mAb but not by methylprednisone. Example 2 describes that development of tactile allodynia was reduced by anti-CD11d mAb treatment. Example 3 discloses that areas of compact myelin after spinal cord injury are increased in animals receiving anti-CD11d mAb treatment. Example 4 discloses that areas of intact neurofilament after spinal cord injury are increased in animals receiving anti-CD11d mAb treatment. Example 5 describes treatment with methylprednisone in combination with anti-CD11d after spinal cord injury (“SCI”); Example 6 describes the effects of treatment with the monoclonal antibody to CD11d on serotonergic innervation of the injured spinal cord. Example 7 describes treatment of inflammatory pain with monoclonal antibody to CD11d in a rat model of disease. Example 8 describes treatment of chronic pain in human patients using anti-CD 11d mAb.

### EXAMPLE 1

### Locomotor Function after Spinal Cord Injury was Improved by an Anti-CD11d mAb but not by Methylprednisone

After central nervous system (CNS) trauma, the immune response involves a mixture of invading neutrophils, natural killer cells and phagocytic monocytes/macrophages [Means et al., J. Neuropathol. & Exp. Neurol. 42:707-719 (1983)]. This response includes the release of inflammatory mediators, induction of reactive microglia, infiltration of platelets, endothelial damage with enhanced vascular permeability and development of edema. Recent observations suggest that post-traumatic inflammation in the spinal cord contributes to chronic CNS deficiencies, partly through demyelination or through more direct damage to neurons and axons [Blight, Central Nervous System Trauma 2:299-315 (1985)].

Both neutrophils and macrophages phagocytose debris which induces an oxidative burst resulting in the production of reactive oxygen species (e.g., nitric oxide, NO— and superoxide, O₂—), which can lead to damage in surrounding healthy tissue. Evidence shows that blocking neutrophil or macrophage infiltration into the CNS using the macrophage toxin, silica, leads to decreases in the extent of injury following stroke or spinal cord injury [Blight, Neuroscience, 60:263-273 (1994)]. Recent studies demonstrated that treatment of animals with monoclonal antibodies that that bind to and block the CD11d subunit [Van der Vieren et al., (1999), supra; Grayson et al., Int. Arch. Allergy Immunol. 118:263-264, 1999] substantially decreased the numbers of neutrophils and macrophages at the site of spinal cord injury [Mabon et al., Exp. Neurol. 166:52-64 (2000); Saville et al, J. NeuroImmunology 156: 42-57 (2004); U.S. Patent No. 6,432,404].

Current treatments for SCI have not effectively addressed the processes that cause secondary complications, such as chronic pain or autonomic dysreflexia, after the initial trauma. To assess the effects of CD11d monoclonal antibodies in reducing symptoms of chronic pain, rats exhibiting spinal cord injury were treated with anti-CD11d antibody.

All animal procedures were done following the Canadian Guide to Care and Use of Experimental Animals. Male Wistar rats (Harlan Bioproducts, Indianapolis, Ind.) were used to study tactile allodynia (chronic pain) and housed in pairs. Rats were anaesthetized as described previously [Weaver et al., J. Neurotrauma 18:1107-1119 (2001)]. The T4 or T12 spinal cord segment was exposed by a dorsal laminectomy and injured, without disrupting the dura, by 60 seconds of clip compression [Weaver et al., supra]. A 50 g calibrated clip (Toronto Western Research Institute, University of Toronto) was used at T4 to induce severe, injury, and a 35 g clip was used at T12 to induce a less severe injury, producing models of autonomic dysreflexia and tactile allodynia, respectively [Bruce et al., Exp. Neurol. 178:33-48 (2002); Weaver et al., supra]. Post-operative care, such as administration of food, water and antibiotics, was provided as described in Weaver et al. [supra].

Rats were blindedly assigned to one of three groups. One of the following treatments was administered intravenously via the tail vein in three consecutive doses at 2, 24 and 48 hr after SCI. The control groups received normal saline (2-week study) or an isotype-matched irrelevant antibody (1B7, 1 mg/kg, 6- and 12-week studies), a second group received the anti-CD11d mAb (Antibody 217L, 1.0 mg/kg) and a third group received methylprednisone (MP) (30 mg/kg at 2 hr and 15 mg/kg at 24 and 48 hr, SOLU-MEDROL®, Pharmacia, Peapack, New Jersey). The dosing regime of MP was chosen because it had effects on intraspinal leukocyte infiltration similar to those of the anti-CD11d mAb. All aspects of the testing and data analysis were done using a blinded experimental design.

Locomotor performance after clip-SCI at T12 was assessed for 12 weeks using the twenty-one point Basso, Beattie and Bresnahan (BBB) scale [Basso, et al., J. Neurotrauma 12:1-21 (1995)]. Locomotor function of the animals was assessed by two independent observers using the BBB open field locomotor score at one day to 12 weeks after SCI. In addition, motor testing after injury at T12 included the inclined plane test and grid-walking [Rivlin et al., J. Neurosurg. 47:577-581 (1977); Kunkel-Bagden et al., Exp. Neurol. 119:153-64 (1993)]. BBB scores of 1-7 indicate increasing movement of the three hind limb joints. A score of 8 indicates sweeping of the hind limbs with no weight support and a score of 10 indicates development of more complex motor control including consistent weight-supported plantar stepping. The inclined plane test determines the ability of the rat to hold its position on an inclined plane with fore and hind limbs.

After the T12 incomplete injury, locomotor improvement was rapid in control rats, and BBB scores stabilized at about 2 weeks, reaching a maximum score of 8±0.2 points. In contrast, BBB scores of the anti-CD11d-treated rats continued to improve after this time and stabilized at about 5 weeks, reaching a score of 1010.5 points. The significantly higher scores of the mAb-treated rats starting at 4 weeks were notable since they were able to execute weight-supported stepping whereas the control rats could only sweep with their hind legs. The BBB scores of MP-treated rats were almost identical to those of the control rats given isotype-matched, irrelevant antibody.

In the inclined plane test, the anti-CD11d-treated rats were able to maintain their position on a tilted platform at a significantly higher angle of incline than the control rats. At 5 weeks after SCI, mAb-treated rats could maintain an angle of approximately 42 degrees while both the MP-treated and control animals only maintained themselves at an angle of approximately 37 degrees. Throughout the course of treatment, the MP-treated rats were not consistently different from the isotype-matched, irrelevant antibody controls.

At 6-12 weeks after T12 SCI, the rats were tested by a grid-walking task [Kunkel-Bagden et al., supra] on a 30 cm long grid with 4 cm square spaces. All of the seven control rats were unable to perform this task and dragged their hind limbs across the grid. From 8-12 weeks after SCI, three of the nine anti-CD11d mAb-treated rats accurately placed their hindpaws on the bars of the grid with an average score of 5±1 footfalls per trial. Only one of the seven MP-treated rats was able to perform this task with 7±0 footfalls per trial.

Spinal cord injury at T4 caused more severe paralysis with a less rapid course of recovery. Beginning at one week post injury, the anti-CD11d mAb-treated rats demonstrated higher BBB scores (score=approximately 3) than the MP-treated rats (score=2) and they had higher scores than the control or MP-treated rats at the 5^{th} and 6^{th} weeks of the study. Anti-CD11d mAb-treated rats reached a plateau of performance at 5 weeks with a score of 8.4±0.3, indicating that they could make sweeping movements with their hind legs whereas the control and MP-treated rats reached scores of 7.4±0.3 and 7.0±0.3, respectively, and could not sweep their hind legs.

These results indicate that treatment with anti-CD11d antibodies moderates the severe locomotor damage characteristic of spinal cord injury, and likely reduces the severity of secondary damage associated with the spinal cord trauma.

### EXAMPLE 2

### Development Of Tactile Allodynia Was Reduced By Anti-CD11d mAb Treatment

To determine the efficacy of anti-CD11d monoclonal antibodies in reducing secondary spinal cord damage and alleviating neuropathological conditions associated with this injury, a rat model of chronic pain was used.

Tactile allodynia was assessed on the dorsal trunk and hind paws before and at 2-12 weeks after SCI at T12. Using a 15 mN modified Semmes Weinstein monofilament, rats were tested once weekly for their response to tactile stimulation of their dorsal trunk area and every second week to stimulation of the hind paws as described in Bruce et al. [supra]. The monofilament used is a single strand of nylon, which has the property of producing a characteristic downward force when buckled on a surface.

At-level, segmental pain is defined as occurring at the transitional zone between normal sensation and sensory loss, generally within a band of two to four spinal segments rostral to the lesion site [Siddall et al., Spinal Cord 39: 63-73 (2001)]. To induce and assess at-level pain, a Modified Semmes-Weinstein filament calibrated to generate an innocuous force of 15 mN, was used to stimulate the dermatome areas corresponding to the 9th-11th thoracic spinal segments. Rats were acclimated to a open cage environment for 20 min and then stimulated ten times at random points within the dorsal trunk area. Each stimulus lasted 3 sec and was separated by a 5 sec interim period. The number of avoidance responses elicited from ten stimulations was then tabulated. Testing resumed seven days following injury with two testing sessions per week for the subsequent four post-injury weeks.

Below-level pain was tested on the plantar surface of the hindpaw. Prior to SCI, rats were acclimated to a Plexiglas chamber (8×3, 5×3, 5 in.) consisting of plastic, mesh walls and an elevated, mesh floor. Similar to dorsal trunk testing, a testing session consisted of ten stimulations to the plantar surface of the hindpaw. Following the testing of one hindpaw, a 2 minute interim period lapsed before the second hindpaw was tested. The number of withdrawal responses for each hindpaw was tabulated and the mean number of withdrawals within both hindpaws was calculated. Testing resumed seven days following injury with two paw testing sessions per week for the subsequent four post-injury weeks. These testing sessions occurred on alternate days ensuring that no animal was tested twice on the same day.

Flinching, escape, paw withdrawal and/or licking, vocalization and biting at the filament indicated that the rat perceived the stimulus as noxious.

Before SCI, the rats rarely exhibited avoidance responses to stimulation with the filament. After SCI, the control rats each showed increasing numbers of avoidance responses to the ten stimuli applied to the dorsal trunk (immediately rostral to the injury) or paws, consistent with the development of tactile allodynia (Bruce et al., supra). Two weeks post injury, control animals receiving isotype-matched, irrelevant antibody exhibited avoidance responses during trunk stimulation with approximately 2-times the frequency of mAb-treated animals, i.e., approximately 4/10 stimuli for control animals compared to < 2/10 stimuli for treated animals. By 12 weeks post injury CD11d mAb treated animals still showed approximately 2-3.5 avoidance responses per 10 stimuli while control animals demonstrated avoidance responses approximately 6-8 avoidance response per 10 stimuli. The anti-CD11d mAb treatment significantly decreased the frequency with which these avoidance responses occurred, reducing the tactile allodynia for the duration of the study. Treatment effects of MP on responses to stimulation of the trunk and paws were inconsistent, but generally demonstrated less relief from tactile allodynia than anti-CD11d mAb treatment.

### EXAMPLE 3

### Areas of Compact Myelin after SCI are Greater with Anti-CD11d mAb Treatment

The improved motor function of CD11d mAb-treated animals indicated that the treatment regimen may be affecting neurons and neuronal cell function and the integrity of glia and oligodendrocytes, which produce myelin for the myelin sheath. To assess neuronal integrity, levels of myelin and neurofilament were assessed at the site of injury and the area surrounding the injury.

At 2, 6 or 12 weeks after SCI, the rats were perfused with 4% formaldehyde and the spinal cord was removed. The cord was cryostat-sectioned transversely in 20 μm sections and serially thaw-mounted on alternate slides. One set of sections was processed for Luxol Fast Blue (2-week study) or solochrome cyanin (6- and 12-week study) staining to identify tightly packed myelin [Weaver et al., supra; Page et al., J. Med. Lab. Tech. 22:224 (1965)] and a second, adjacent set was immunoprocessed for staining with neurofilament 200 to assess axon integrity [Bruce et al., supra].

Digitized images of every eighth section on a slide were collected and, using a calibrated function of Image Pro Plus software, the stained areas were quantified. The averaged area of five sections was calculated and plotted as a sample at 0.4 mm lengths along the cord. The smallest average area was considered the epicenter of the lesion. The data were normalized as described below, after first establishing that the cord cross-sectional area 3 mm rostral and caudal to the T4 or Ti 2 dorsal roots is uniform in the intact spinal cord.

After the T12 or T4 SCI, compact myelin at the lesion epicenter of control rats was barely detectable. As shown in samples of spinal cord sections at 6 weeks after SCI at T4, myelin in the lesion epicenter appeared as small punctate areas of dark blue staining that may have surrounded axons. At 1.5-2.0 mm caudal to the injury at T4 or T12 this dark blue myelin staining encompassed much of the white matter. More myelin was visible beginning 1.0-1.5 mm from the lesion epicenter in rats treated with the anti-CD11d mAb compared to isotype-matched control rats. This difference persisted and increased with distance from the lesion center. The differences were significant when assessed at 2, 4, 6, and 12 weeks after SCI.

Areas of staining were determined by quantitative morphometry, and the area in each rat expressed as a percent of the total cross-sectional area of the relatively intact thoracic cord sampled 3-4 mm rostral to the epicenter of the injury. Areas of myelin in the anti-CD11d mAb-treated animals were significantly greater than those in control rats at most cord levels up to 3.2 mm from the T12 or T4 injury site. After the T12 injury and at 6 weeks after the T4 injury, the MP treatment also led to larger areas of myelin.

These results demonstrate that treatment with anti-CD 11d mAb promotes myelin sparing, or regeneration, in injured animals. This increase in myelination of neurons promotes more efficient neuronal signal transmission, and suggests that anti-CD11d mAb treatment presents an effective therapy for the treatment of neuronal transmission and decrease the symptoms of chronic pain.

### EXAMPLE 4

### Areas of Intact Neurofilament Fibers after SCI are Greater with Anti-CD11d mAb Treatment

Serial sections of spinal cord adjacent to those stained for myelin were processed for neurofilament 200-immunoreactive protein to determine if the larger areas of myelin were paralleled by greater areas containing intact axons.

In cord sections at 6 weeks after T4 SCI, bundles of neurofilament at the lesion epicenter were arrayed irregularly in the white and grey matter and isolated areas of the neuropil appeared intact. Punctuate areas of staining revealed axon bundles in cross section. Within the lesion, neurofilament fibers were mostly arrayed in a disorganized pattern in isotype-matched irrelevant antibody control and CD11d mAb-treated rats but fields of intact neurofilament were more frequently found in the mAb-treated rats. The lesion sites and sections 1.0 mm-2.0 mm from the lesion epicenter of rats treated with the anti-CD11d mAb had more regions with regularly dispersed white matter axon bundles than the control rats, and significantly increased levels of intact neurofilament.

At 12 weeks after SCI at T12, the spinal cord within 2 mm of the epicenter was significantly damaged. The neurofilament staining confirmed a larger, longer cavitation at the T12 injury site than at the T4 site. In contrast to the greater myelin in the T12 region after anti-CD11d treatment, the areas of intact neurofilament were not different from those of the control rats in all areas. However, neurofilament areas were significantly greater in rats treated with the anti-CD11d mAb at the lesion epicenter and at 1.0 mm and 2.0 mm from the center. No assessments were made at greater distances. Effects of MP on neurofilament sparing were similar to those of the anti-CD11d mAb with the exception that MP had no significant effect on neurofilament in the T12 lesion epicenter at 12 weeks after SCI. After MP treatment, the area of neurofilament at the T12 lesion epicenter was significantly less than that of control rats. In contrast, at 6 weeks after the T4 injury, the areas of intact neurofilament at, and caudal to, the lesion epicenter were significantly larger after anti-CD11d mAb treatment than those in control rats. Intact neurofilament areas in rats treated with MP were not different from those in control rats.

The increase in neurofilament density in injured animals treated with anti-CD11d antibody indicates that blocking CD11d promotes axon regeneration or sprouting and/or prevents degeneration, and again improves neuronal signal transmission. Thus, the increase in both myelin deposit and axon filament growth and/or integrity in the nervous system of spinal cord injured subjects treated with anti-CD11d mAb suggests that anti-CD11d mAb treatment is an effective therapy to alleviate symptoms of chronic pain derived from neuronal damage.

### EXAMPLE 5

### Treatment with Methylprednisone in Combination with the Anti-CD11d Abolishes the Positive Neurological Benefits of the Anti-CD11d Treatment

Because methylprednisone is such a potent immunosuppressant, and has been shown to be effective in treatment of spinal cord injury [Bracken, Spine. 26 (24 Suppl):S47-54 (2001)], a study was done to test the effects of combining the MP treatment with the anti-CD11d antibody treatment.

Animals were treated with either an isotype-matched irrelevant antibody (1B7 mAb, 1 mg/kg), anti-CD11d mAb (1.0 mg/kg) or MP following the injection schedules described above at 2, 24 and 48 hr after SCI. MP (SOLU-MEDROL®, Upjohn, Peapack, N.J.) was given at 2-hr (30 mg/kg), 24 and 48 hr (15 mg/kg) post-SCI. The combined treatment group received both anti-CD11d mAb and MP, delivered simultaneously following the protocol described above. The anti-CD11d mAb and MP were diluted to a final volume of 100-180 μL in phosphate-buffered saline, pH 7.2, lacking calcium and magnesium (Invitrogen, Burlington, ON, CA). For the combined delivery of anti-CD11d mAb and MP, concentrations were adjusted to permit a 200 μL injection volume. All aspects of the testing and data analysis were done using a blinded experimental design.

At two weeks after SCI, BBB locomotor scores in the combined treatment group (n=4) was not significantly different from the control group (n=4). The BBB score of the anti-CD11d mAb-treated group was ˜6 while the MP-treated group had a score of ˜3. At six weeks after SCI, effects on autonomic dysreflexia were assessed; The increases in arterial pressure caused by distension of the colon were approximately 30 mmHg in the control group, 22 mmHg in the anti-CD11d mAb-treated group, 24 in the MP-treated group, and 30 mmHg in the group treated with the mAb and MP. At two weeks after SCI, the areas of myelin caudal to the injury epicenter were significantly greater in the anti-CD11d mAb treatment group. These areas in the MP and combined treatment groups were almost identical to those in the control group.

These findings demonstrate that combination of the anti-CD11d mAb treatment with MP leads to loss of the positive treatment effects of the anti-CD11d monoclonal antibody.

### EXAMPLE 6

### Treatment with Monoclonal Antibody to CD11d Modulates Serotonergic Innervation of the Injured Spinal Cord and Improves Recovery

Sensory, motor and autonomic disorders resulting from SCI correspond with defined alterations in the density and distribution of serotonin (5-HT) fibers surrounding the lesion site [Bruce et al., Exp. Neurol. 178:33-48 (2002); Hains et al., Exp. Neurol. 175:347-362 (2002)]. Spinal cord injury (SCI) leads to loss of descending serotonergic (5-hydroxytryptamine; 5-HT) control of several populations of spinal neurons. Spinal serotonergic projections from the brainstem synapse in the dorsal horn, causing inhibition or facilitation of pain signaling, depending upon the receptor stimulated [Calejesan et al., Brain Res. 798:46-54 (1998); Bardin et al., Eur. J. Pharmacol. 409:37-43 (2000)]. Serotonergic neurons also target sympathetic pre-ganglionic neurons in the intermediolateral cell column (IML), contributing to autonomic regulation [Allen et al., J Comp Neurol. 350:357-366 (1994); Jacobs et ale, Brain Res. Rev. 40:45-52 (2002)]. Descending serotonergic axons also provide excitatory input to ventral horn α-motoneurons [Saruhashi et al., Exp. Neurol. 139: 203-213 (1996)]. Thus, loss of descending-serotonergic inputs caudal to (below) the lesion site contributes to neuropathic pain and motor dysfunction after spinal cord injury [Saruhashi et al., supra; Hains et al., supra].

In addition to loss of serotonergic axons below the lesion level, a marked increase in the area of serotonergic-immunoreactivity (5-HT-Ir) occurs in the dorsal horn of spinal segments immediately rostral to (above) the injury [Bruce et al., supra; Inman et al., J. Comp. Neurol. 462:431-449 (2003)]. These segments immediately rostral to (above) the injury correspond with dermatomes in which a band of tactile/mechanical allodynia often develops [Tasker R., The Management of Pain, (Bonica J J, ed), pp 264-283. Philadelphia: Lea & Febiger (1990); Vierck, Jr. et al., Pain 89:1-5 (2000)]. This at-level pain can be attributed to the increased serotonin as it is blocked by the 5-HT₃ receptor antagonist ondansetron [Oatway et al., Pain 110:259-268 (2004)]. In contrast, neuropathic pain caudal to a SCI can be due to loss of serotonergic actions on 5-HT₁ and 5-HT₂ receptors [Hamon et al., Novel Aspects of Pain Management: Opioids and Beyond (Sawynok J, Cowan A, eds), pp 203-228, Wiley-Liss, Inc. (1999); Hains et al., supra]. Therefore plasticity of descending serotonergic fibers above an injury, and their loss below the injury, both contribute to neuropathic pain after spinal cord injury.

Anti-CD11d mAb Treatment Modulates Dorsal Horn Serotonergic Fiber Density Rostral and Caudal to Lesion

It is believed that the anti-CD11d mAb treatment decreases chronic pain and improves motor and autonomic function, in part, by preventing the increases and decreases, respectively, in serotonergic input to neurons rostral and caudal to a moderate SCI. To confirm this hypothesis, the density and distribution of 5-HT-immunoreactive fibers in the dorsal, intermediate and ventral horns was assessed in sham-injured, vehicle-treated and anti-CD11d-treated rats.

To assess the density and distribution of the fiber immunoreactivity, randomly selected floating, transverse sections (T9-11, L2-4) were mounted on slides and viewed by light and fluorescence microscopy using a Leica microscope (Leica, Canada). Digitized images, captured using a DAGE videocamera (MTI, Michigan City, Ind.) for bright-field images and a Retiga 1300 camera (Q Imaging, Burnaby, BC) for fluorescent images, were collected and processed using Image Pro Plus software (Media Cybernetics, Silver Spring, Md.). Image processing software was used to normalize all images to a common pixel intensity range, providing an equalized comparison as, previously described [Bruce et al., supra].

Quantification of the dorsal horn 5-HT-Ir was performed by visually selecting an area of interest encompassing laminae I-IV for each individual image. The total area of interest was 136 mm² for sections within the T9-11 segment and 272 mm² for L2-4 sections. For each segment, 20-25 sections per animal were quantified by randomly selecting one section per row of eight on a slide.

Four weeks following SCI at T12, the distribution and density of 5-HT fibers within laminae I-IV of the dorsal horn, where nociceptive information begins to be processed in the central nervous system, were examined both immediately rostral (T9-T11) and caudal (L2-L4) to the lesion site.

5-HT-Ir within laminae I-IV of sham-injured animals consisted of punctate fibers, primarily within the superficial laminae. In sham-injured rats, the area of 5-HT-Ir in segments T9-11 was 4984±841 μm² (n=6). The area of 5-HT-Ir was significantly increased in the rostral segments (T9-11) to 17761±1014 μm² in the vehicle-treated SCI group (n=5, P<0.05). This approximately four-fold increase was most apparent within the superficial laminae and was accompanied by an increased number of fibers spread throughout laminae III and IV. Following anti-CD11d mAb treatment, 5-HT fiber distribution in T9-11 appeared normalized within the superficial laminae. The area of 5-HT-Ir at T9-11 following anti-CD11d treatment, 7884±516 μm² (n=6), was significantly less than that in the vehicle-treated group (P<0.05).

The distribution of 5-HT-Ir in segments caudal to the injury (L2-4) in the sham-injured group was similar to that in T9-11. The area of 5-HT-Ir in laminae I-IV in this group was 14000±756 μm² (n=5). In a majority of cases, 5-HT-Ir was completely lost caudal to the SCI in vehicle-treated SCI rats, with detectable immunoreactivity in L2-4 significantly decreased to 589±159 μm² in these animals (n=5, P<0.05). After treatment with the anti-CD11d mAb, tortuous, punctate fibers were distributed randomly throughout the superficial laminae. In this group of rats, the area of 5-HT-Ir in L2-4 (3842±1190 μm², n=5) was significantly increased following anti-CD11d mAb treatment when compared to that after vehicle treatment (P<0.05).

These results demonstrate that fiber density rostral to the injury was similar to normal levels in the anti-CD11d-treated rats, and the density of these fibers was increased caudal to the lesion after anti-CD11d treatment, coinciding with reductions in mechanical allodynia, at and below the injury, respectively.

Anti-CD11d mAb Treatment Increases Serotonergic Fiber Distribution in the Intermediolateral Cell Column Caudal to Lesion

To investigate whether the modulation of 5-HT fiber density and distribution observed in the dorsal horn following anti-CD11d mAb treatment was consistent throughout other laminae within the same segments, the area of 5-HT-Ir was quantified in the intermediolateral cell column (IML). Assessment of fiber density was performed as described above for the dorsal horn. The total area of interest quantified for the IML in T9-11 and L2-4 sections was 208 mm².

In the T9-11 segments of sham-injured animals, 5-HT-Ir appeared in long and varicose fibers clustered in the IML and spreading medially and laterally, with an area of 3331±495 μm² (n=6). The areas of immunoreactivity in the segments rostral to the lesion site were not significantly altered following vehicle treatment (3944±797 μm², n=5)) or anti-CD11d mAb treatment (4662±324 μm², n=6). In the vehicle- and mAb-treated groups, the distribution and morphology of 5-HT-Ir fibers were similar to those observed in the sham-injured animals.

In segments caudal to the lesion (L2-4), the distribution of 5-HT-Ir fibers in the IML of sham-injured rats was similar to that described for the thoracic segments. The area of 5-HT-Ir in sham-injured rats was 2891±683 μm² (n=6). In contrast, four weeks following SCI, 5-HT-Ir at L2-4 was almost completely eliminated in vehicle-treated animals, and was significantly decreased to 310±125 μm² (n=5, P<0.05). Following anti-CD11d mAb treatment, an increase in 5-HT-Ir within the IML was clearly visible. The area of 5-HT-Ir at L2-4 in the treated group (5848±1373 μm², n=6) was significantly increased when compared to that in both the sham-injured and vehicle-treated groups (P<0.05).

Blockade of CD11d inflammatory signals in SCI animals led to increased 5-HT-Ir fiber density in the IML caudal to injury, contributing to an increase in autonomic recovery. These results indicate that treatment of spinal cord injury with anti-CD11d antibody can significantly enhance the recovery of serotonergic reactivity, and enhances the ability of neurons to signal after spinal cord injury.

Anti-CD11d mAb Treatment Protects Serotonergic Fiber Distribution in the Ventral Horn Caudal to Lesion

Descending serotonergic axons provide excitatory input to ventral horn α -motoneurons [Saruhashi et al., supra]. To determine the effects of anti-CD11d treatment on the ventral horn, assessment of fiber density was performed as described above for the dorsal horn and IML. The total area of interest quantified for the ventral horn in L2-4 segment was 290 mm². The ventral horn area of interest encompassed laminae VII-IX and care was taken to exclude any fibers from the IML, to avoid redundant quantification.

As was found in the IML, the distribution and density of 5-HT-Ir fibers around the small number of motor neurons in the ventral horn rostral to the lesion were not noticeably altered by SCI or anti-CD11d mAb treatment at four weeks after SCI. In contrast, marked changes in ventral horn 5-HT-Ir caudal to SCI were obvious. In the sham-injured group, numerous 5-HT immunoreactive fibers were observed throughout the ventral horn. Immunoreactive product also appeared within terminal boutons surrounding the somata of motoneurons, particularly in sham-injured and anti-CD11d mAb-treated animals.

The area of ventral horn 5-HT-Ir in segments L2-4 of sham-injured animals was 18565±2019 μm² (n=5). Four weeks post-injury, beaded, varicose fibers immunoreactive for 5-HT were sparsely distributed throughout the ventral horn in vehicle-treated rats. The area of 5-HT-Ir in the vehicle treated animals (1255±363 μm²; n=6, P<0.05) was significantly smaller than that in sham-injured animals. This decrease in 5-HT-Ir fibers following SCI was partially reversed by anti-CD11d mAb treatment. Quantification of the areas of 5-HT-Ir fibers revealed a significant, 10-fold increase following anti-CD11d treatment (11882±1220 μm²) (P<0.05) compared to vehicle-treated animals.

The treatment led to increased 5-HT-Ir fiber density in the ventral horn caudal to injury. Increased 5-HT fiber density within the lumbar ventral horn, caudal to the lesion site, following anti-CD11d mAb treatment may therefore be directly related to the recovered locomotion via modulated excitatory input to α-motoneurons and central pattern generator circuitry. These changes provide increased autonomic recovery and correlate with improved locomotor function.

5-HT-Ir Fibers Caudal to the Lesion Site Appear to Originate from Spared Axons Following Anti-CD11d mAb Treatment

Previous studies have shown that greater axonal sprouting may be associated with grey and white matter sparing adjacent to the site of injury [Gris et al., J. Neurosci. 24:4043-4051 (2004)]. An increase in intact grey matter provides a larger number of denervated neuronal targets, prompting sprouting of axons into areas below the injury site [Polistina et al., J. Comp. Neurol. 299:349-363 (1990)].

To better visualize and compare the sprouting of 5-HT fibers within and surrounding the lesion, longitudinal sections encompassing the entire lesion were analyzed. Based on tissue integrity, the rostral and caudal borders of the lesion site were easily identified. Cyst formation within the lesion site was characteristic in both vehicle and anti-CD11d mAb-treated animals. In vehicle-treated rats, a dense accumulation of tortuous 5-HT-Ir fibers occurred rostral to the lesion site. These fibers did not penetrate through the lesion site border. Clusters of 5-HT-Ir fibers coursed along the sub-pial rim of the cord, on the lateral edge of the lesion site, continuing through to the caudal border. A few individual 5-HT-Ir fibers were sparsely dispersed within the gray matter in random patterns caudal to the lesion site.

Following anti-CD11d mAb treatment, descending 5-HT-Ir fibers still accumulated at the rostral border of (above) the lesion site. However, clusters of 5-HT-Ir fibers extended along the lateral edge of the cord passing through both the rostral and caudal borders of the lesion site. Adjacent to the rostral part of the lesion, these fiber-bundles on the side of the spinal cord appeared thicker in the anti-CD11d-treated rats than in the vehicle-treated rats. Caudal to (below) the lesion site, the beaded, 5-HT-Ir fibers continued along one side of the cord, at the sub-pial rim, and appeared to send collateral branches medially through the white matter, toward the grey-white matter border where a large proportion of 5-HT-Ir fibers were distributed within the IML. The area of 5-HT-Ir fibers in the lesion or in the subpial rim adjacent to the lesion of anti-CD11d mAb-treated rats was more than 10-fold larger than the areas in vehicle-treated rats. This may be attributed, in part, to thicker bundles coursing along the subpial edge of the rostral part of the lesion.

These results demonstrate that the number of sprouting collaterals from bulbospinal axons was enhanced following anti-CD11d mAb treatment, which may provide enhanced supraspinal connectivity.

Anti-CD11d mAb Treatment does not Increase the Number of Raphe-Spinal Axons Traversing the Lesion Site

Spinal serotonergic projections from the nucleus raphe magnus (NRM) cause inhibition or facilitation of pain signaling [Calejesan et al., supra; Bardin et al., supra], while serotonergic neurons within the nuclei raphe obscurus (NRO) and pallidus (NRP) target sympathetic preganglionic neurons in the intermediolateral cell column (IML), contributing to autonomic regulation [Allen and Cechetto, supra; Jacobs et al., supra]. Loss of these descending serotonergic inputs caudal to the lesion site contributes to neuropathic pain and motor dysfunction after SCI [Saruhashi et al., supra; Hains et al, supra], and potentially to autonomic dysfunction. Additionally, transplantation of 5-HT-releasing embryonic raphe cells following transection SCI leads to locomotor improvement [Ribotta et al., J Neurosci 20:5144-5152 (2000)], implying that these cells play an important role in the central nervous system.

To assess the effects of anti-CD11d mAb treatment on raphe-spinal axons after clip compression, retrogradely transported FLUOROGOLD® was used to assess the integrity of raphe-spinal axons projecting through the epicenter and caudal to the spinal cord lesion site four weeks following moderate clip-compression injury. The retrograde tracing method used to label cell bodies within the raphe nucleus has been described previously [Fehlings et al., Exp. Neurol. 132:220-228 (1995); Joshi et al., J Neurotrauma 19:191-203 (2002)]. A 3 mm×3 mm Gelfoam pledget (Pharmacia, Mississauga, ON) was used to absorb 7.5 μl of 4% FLUOROGOLD® (Hydroxystilbamidine; Fluorochrome Inc., Englewood, Colo.). The Gelfoam pledget was placed at the transection site against the proximal end of the cord. Dispersion of the FLUOROGOLD® was minimized by applying petroleum jelly over the transection site. The rats survived for seven days following transection to allow the tracer to be transported to the medulla. To ensure that the FLUOROGOLD® traveled through the lesion site and to the medulla via retrograde axonal transport and not by passive dispersion through the cerebrospinal fluid, a control injury was used in which the cord was transected at the 12th and 13th thoracic segments junction rather than compressed (n=3). The tracer was then applied at the 4th lumbar segment as previously described.

Comparisons were made between the neuron counts in the nuclei raphe magnus and raphe pallidus in the anti-CD11d mAb treated group and the vehicle group. The FLUOROGOLD®-labelled neurons in both treatment groups were similar in physical structure and appeared as triangular or oval, multipolar cell bodies approximately 15-30 μm in size. Many of the FLUOROGOLD®-labelled raphe neurons were immunoreactive for 5-HT. Cell counting revealed no significant difference between the mean number of labelled neurons within the raphe nucleus of the anti-CD11d mAb-treated (n=6) and the saline-treated groups. The mAb-treated group had significantly greater variance than the saline-treated group (P<0.05), due to high cell counts in some of the anti-CD11d-treated rats.

Treatment with anti-CD11d appeared to improve the collateral sprouting of spared axons caudal to the injury site rather than significantly increasing the number of axons traversing through the lesion. Overall, these results demonstrate that improved motor and sensory recovery following early treatment with the anti-CD11d mAb is associated with a more normal pattern of 5-HT serotonergic fiber distribution both rostral and caudal to the SCI lesion site and with tissue sparing at and near the lesion epicenter.

The complex serotonergic influences on pain and motor function are greatly disrupted by SCI. This anti-inflammatory strategy provides a significant and effective method for reversing the damaging effects of spinal cord injury. These results demonstrate that the tissue sparing caused by targeting the early inflammatory cascade after SCI results in a more characteristic distribution of serotonergic fibers rostral and caudal to the injury, associated with improvements in sensations of chronic pain.

### EXAMPLE 7

### Treatment of Inflammatory Pain with Monoclonal Antibody to CD11d

Results described in the above examples demonstrated that anti-CD11d monoclonal antibodies provided effective means for alleviating symptoms of tactile allodynia, one form of chronic pain. Because several other sources of chronic pain exist, anti-CD11d therapy is contemplated for use in inflammatory pain.

To assess the therapeutic effects of anti-CD11d in inflammatory pain, the rat carrageenan paw model is used. Carrageenan is a water-extractable polysaccharide obtained from seaweeds. Injection of lambda carrageenan (a hydrocolloid that does not form a gel) into the plantar foot, or the knee joint, results in a localized inflammation that leads to decreased weight bearing, guarding of the affected limb, and hyperalgesia (See U.S. Pat. No. 6,489,356). Carrageenan-induced hyperalgesia is believed to occur as a consequence of sensitization of primary afferent nociceptors, small nerve endings in the skin and other tissues that respond only to strong stimuli, and neuron plasticity intrinsic to the spinal cord.

Animals are administered by injection an appropriate dose of carrageenan, such as 0.1 ml of a 1% solution in 0.85% saline, or in the range of 2 mg to 6 mg carrageenan in appropriate buffer [Coulthard et al., J. Neurosci. Methods 128:95-102 (2003)]. Baseline measurement of hindpaw withdrawal latency to thermal stimulation were obtained on a Hargreaves device. Animals receive a single injection, e.g., intrathecally (IT), intravenously or intraperitoneally or via any other appropriate route, of anti-CD11d ranging from 0.5 to 40 μg, as appropriate. Withdrawal thresholds are measured on a Hargreaves device every 30 minutes for three hours.

A reduction in hypersensitivity to the inflamed foot to pre-carrageenan levels in anti-CD11d treated animals indicates that CD11d mAb is an effective treatment for inflammatory pain and inflammatory hyperalgesia.

### EXAMPLE 8

### Treatment Of Chronic Pain in Human Patients

Treatment with monoclonal antibodies to the CD11d subunit proved effective in reducing the extensive secondary injury associated with spinal cord injury in rats. It is contemplated that treatment of human subjects suffering from chronic pain resulting from either spinal cord trauma or other source of chronic pain may be treated with anti-CD Id antibodies, pharmaceutical compositions comprising anti-CD11d antibodies, or compositions comprising a polypeptide that specifically binds CD11d.

A composition comprising a polypeptide that specifically binds CD11d is administered to subjects suffering from chronic pain by any route deemed appropriate by the treating physician. In one aspect, it is contemplated that the anti-CD11d antibody/polypeptide therapy is administered intravenously. It is recognized by one of skill in the art that the dose of a polypeptide of the invention that specifically binds CD11d administered to a patient suffering from chronic pain will vary from patient to patient, and may be anywhere from 1 mg/kg/day to 100 mg/kg/day. Polypeptides of the invention are administered in doses appropriate for the patient's size, sex, and weight, as would be known or readily determined in the art. Subsequent doses of the polypeptide may be increased or decreased to address the particular patient's response to therapy.

A polypeptide that specifically binds CD11d is given in any formulation recognized in the art to allow the composition to diffuse into the bloodstream or tissue sites, e.g. aqueous solution or oily suspension. It is contemplated that other agents useful in treating chronic pain are administered in the same formulation as anti-CD11d antibody and given simultaneously. Alternatively, the agents may also be administered in a separate formulation and still be administered concurrently with anti-CD11d antibody. The second agent may also be administered prior to administration of a polypeptide that specifically binds CD11d. Prior administration refers to administration of the agent within the range of one week prior to anti-CD11d antibody/polypeptide treatment up to 30 minutes before administration of a composition comprising a polypeptide that specifically binds CD11d. It is further contemplated that the second agent is administered subsequent to administration of a polypeptide that specifically binds CD11d. Subsequent administration is meant to describe administration from 30 minutes after administration of polypeptide that specifically binds CD11d up to one week after anti-CD11d antibody/polypeptide treatment.

It is contemplated that the anti-CD11d antibody or polypeptide that binds CD11d is administered within 2-72 hours after injury to spinal cord. Additionally, polypeptide that specifically binds CD11d may be administered daily, weekly, bi-weekly, or at other effective frequencies, as would be determinable by one of ordinary skill in the art.

In some instances, treatment of chronic pain in human patients is carried out generally as described in U.S. Pat. No. 6,372,226. A patient experiencing acute inflammatory pain, neuropathic pain, spastic conditions, or other chronic pain from an injury, e.g. spinal cord injury, is treated by intrathecal administration, for example by spinal tap to the lumbar region, with an appropriate dose of a composition described herein for use in a method of the invention. The particular dose and site of injection, as well as the frequency of administrations, depend upon a variety of factors within the skill of the treating physician. Within 1-7 days after administration the patient's pain is substantially alleviated. It is contemplated that efficacy and timing of the pain relief will differ with each patient and may appear later than 7 days after administration of the therapeutic composition.

The compositions described herein can be injected at different spinal levels to treat pain at various sites in the body. Additionally, a catheter can be percutaneously inserted into the intrathecal space via lumbar puncture at vertebral level L3-4 or L4-5 using a Tuohy needle. The catheter can be adjusted to different vertebral locations and/or used at different dose concentrations to treat different types of pain and/or spasm.

Chronic pain is assessed by an objective scaled test such as the Leeds Assessment of Neuropathic Symptoms and Signs (LANSS) Pain Scale [Bennett, M. Pain. 92:147-157 (2001)]. A decrease in hypersensitivity to pain stimulus after treatment with a composition comprising a polypeptide that specifically binds CD11d indicates that interfering with normal activity of the CD11d integrin subunit alleviates symptoms associated with chronic pain. It another aspect of the invention, the compositions described herein are administered in conjunction with another pain medications as described above, wherein the therapies provide a synergistic effect in relieving symptoms of chronic pain.

Numerous modifications and variations in the invention as set forth in the above illustrative examples are expected to occur to those skilled in the art. Consequently only such limitations as appear in the appended claims should be placed on the invention.

What is claimed is:

1. A method for treating chronic pain in a mammalian subject comprising the step of administering to a subject in need a therapeutically effective amount of a composition comprising a polypeptide that specifically binds CD11d.

2. The method of claim 1 wherein the said polypeptide is an antibody.

3. The method of claim 1 wherein said polypeptide is a monoclonal antibody.

4. The method of claim 1 wherein said polypeptide is a monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593).

5. The method of claim 1 wherein the polypeptide comprises one, two and/or three complementarity determining region (CDR) of a light chain of monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593).

6. The method of claim 1 wherein the polypeptide comprises one, two and/or three complementarity determining region (CDR) of a heavy chain of monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593).

7. The method of claim 1 wherein the polypeptide comprises one, two and/or three complementarity determining regions (CDR) of a heavy chain of monoclonal antibody secreted by hybridoma 217L, 226H or 236L and one, two and/or three complementarity determining regions (CDR) of a light chain of monoclonal antibody secreted by hybridoma 217L (American Type Culture. Collection Accession No: HB-12701), hybridoma 226H (American Type Culture Collection Accession No: HB-12592) or hybridoma 236L (American Type Culture Collection Accession No: HB-12593).

8. The method of claim 1 wherein polypeptide recognizes an epitope on CD11d recognized by a monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593).

9. The method of claim 1 wherein the polypeptide competes with a monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593), for binding to CD11d.

10. The method of claim 1 wherein the polypeptide comprises one, two, three, four, five and/or six complementarity determining regions of a monoclonal antibody secreted by hybridoma 217L (American Type Culture Collection Accession No: HB-12701), hybridoma 226H (American Type Culture Collection Accession No: HB-12592) or hybridoma 236L (American Type Culture Collection Accession No: HB-12593), said polypeptide selected from the group consisting of a monoclonal antibody, a polyclonal antibody, a single chain antibody, a chimeric antibody, a bifunctional/bispecific antibody, a humanized antibody, a human antibody, and a complementarity determining region (CDR)-grafted antibody and a peptibody.

11. The method of any one of claims 1 through 10 wherein the mammal is a human.

12. The method of any one of claims 1 through 10 wherein the chronic pain is selected from the group consisting of tactile allodynia, neuropathic pain, hyperalgesia, hyperpathia, and inflammatory pain.

13. The method of claim 12 wherein the chronic pain is tactile allodynia.

14. The method of any one of claims 1 through 10 wherein the chronic pain results from central nervous system trauma or spinal cord injury.

15. The method according to any one of claims 1 through 10 wherein the spinal cord injury is compression of the spinal cord.

16. The method of any one of claims 1 through 10 wherein administration of the composition results in an increase in axon regeneration and/or growth.

17. The method of any one of claims 1 through 10 wherein administration of the composition results in an increase in myelin regeneration.

18. The method of any one of claims 1 through 10 wherein the composition further comprises a pharmaceutically acceptable diluent or carrier.

19. The method of any one of claims 1 through 10 wherein the composition is administered in conjunction with other pain relief medicine.

20. The method of claim 19 wherein the other pain relief medicine is selected from the group consisting of NSAIDs, analgesics, steroids, and anti-epileptic medicines.


SEQUENCE LISTING (2 sequences)

SEQ ID NO: 1 - DNA, 3726 bp, Homo sapiens
  CDS (3)..(3485)
tgaccttcggcactgtgcttcttctgagtgtcctggcttcttatcatggattcaacctggatgtggaggagcctacgatcttccaggaggatgcaggcggctttgggcagagcgtggtgcagttcggtggatctcgactcgtggtgggagcacccctggaggtggtggcggccaaccagacgggacggctgtatgactgcgcagctgccaccggcatgtgccagcccatcccgctgcacatccgccctgaggccgtgaacatgtccttgggcctgaccctggcagcctccaccaacggctcccggctcctggcctgtggcccgaccctgcacagagtctgtggggagaactcatactcaaagggttcctgcctcctgctgggctcgcgctgggagatcatccagacagtccccgacgccacgccagagtgtccacatcaagagatggacatcgtcttcctgattgacggctctggaagcattgaccaaaatgactttaaccagatgaagggctttgtccaagctgtcatgggccagtttgagggcactgacaccctgtttgcactgatgcagtactcaaacctcctgaagatccacttcaccttcacccaattccggaccagcccgagccagcagagcctggtggatcccatcgtccaactgaaaggcctgacgttcacggccacgggcatcctgacagtggtgacacagctatttcatcataagaatggggcccgaaaaagtgccaagaagatcctcattgtcatcacagatgggcagaagtacaaagaccccctggaatacagtgatgtcatcccccaggcagagaaggctggcatcatccgctacgctatcggggtgggacacgctttccagggacccactgccaggcaggagctgaataccatcagctcagcgcctccgcaggaccacgtgttcaaggtggacaactttgcagcccttggcagcatccagaagcagctgcaggagaagatctatgcagttgagggaacccagtccagggcaagcagctccttccagcacgagatgtcccaagaaggcttcagcacagccctcacaatggatggcctcttcctgggggctgtggggagctttagctggtctggaggtgccttcctgtatcccccaaatatgagccccaccttcatcaacatgtctcaggagaatgtggacatgagggactcttacctgggttactccaccgagctagccctgtggaagggggtacagaacctggtcctgggggccccccgctaccagcataccgggaaggctgtcatcttcacccaggtgtccaggcaatggaggaagaaggccgaagtcacagggacgcagatcggctcctacttcggggcctccctctgctccgtggatgtggacagcgatggcagcaccgacctgatcctcattggggccccccattactatgagcagacccgagggggccaggtgtccgtgtgtcccttgcctagggggcagagggtgcagtggcagtgtgacgctgttctccgtggtgagcagggccacccctggggccgctttggggcagccctgacagtgttgggggatgtgaatgaggacaagctgatagacgtggccattggggccccgggagagcaggagaaccggggtgctgtctacctgtttcacggagcctcagaatccggcatcagcccctcccacagccagcggattgccagctcccagctctcccccaggctgcagtattttgggcaggcgctgagtgggggtcaggacctcacccaggatggactgatggacctggccgtgggggcccggggccaggtgctcctgctcaggagtctgccggtgctgaaagtgggggtggccatgagattcagccctgtggaggtggccaaggctgtgtaccggtgctgggaagagaagcccagtgccctggaagctggggacgccaccgtctgtctcaccatccagaaaagctcactggaccagctaggtgacatccaaagctctgtcaggtttgatctggcactggacccaggtcgtctgacttctcgtgccattttcaatgaaaccaagaaccccactttgactcgaagaaaaaccctgggactggggattcactgtgaaaccctgaagctgcttttgccagattgtgtggaggatgtggtgagccccatcattctgcacctcaacttctcactggtgagagagcccatcccctccccccagaacctgcgtcctgtgctggccgtgggctcacaagacctcttcactgcttctctccccttcgagaagaactgtgggcaagatggcctctgtgaaggggacctgggtgtcaccctcagcttctcaggcctgcagaccctgaccgtggggagctccctggagctcaacgtgattgtgactgtgtggaacgcaggtgaggattcctacggaaccgtggtcagcctctactatccagcagggctgtcgcaccgacgggtgtcaggagcccagaagcagccccatcagagtgccctgcgcctggcatgtgagacagtgcccactgaggatgagggcctaagaagcagccgctgcagtgtcaaccaccccatcttccatgagggctctaacggcaccttcatagtcacattcgatgtctcctacaaggccaccctgggagacaggatgcttatgagggccagtgcaagcagtgagaacaataaggcttcaagcagcaaggccaccttccagctggagctcccggtgaagtatgcagtctacaccatgatcagcaggcaggaagaatccaccaagtacttcaactttgcaacctccgatgagaagaaaatgaaagaggctgagcatcgataccgtgtgaataacctcagccagcgagatctggccatcagcattaacttctgggttcctgtcctgctgaacggggtggctgtgtgggatgtggtcatggaggccccatctcagagtctcccctgtgtttcagagagaaaacctccccagcattctgacttcctgacccagatttcaagaagtcccatgctggactgctccattgctgactgcctgcagttccgctgtgacgtcccctccttcagcgtccaggaggagctggatttcaccctgaagggcaatctcagtttcggctgggtccgcgagacattgcagaagaaggtgttggtcgtgagtgtggctgaaattacgttcgacacatccgtgtactcccagcttccaggacaggaggcatttatgagagctcagatggagatggtgctagaagaagacgaggtctacaatgccattcccatcatcatgggcagctctgtgggggctctgctactgctggcgctcatcacagccacactgtacaagcttggcttcttcaaacgccactacaaggaaatgctggaggacaagcctgaagacactgccacattcagtggggacgatttcagctgtgtggccccaaatgtgcctttgtcctaataatccactttcctgtttatctctaccactgtgggctggacttgcttgcaaccataaatcaacttacatggaaacaacttctgcatagatctgcactggcctaagcaacctaccaggtgctaagcaccttctcggagagatagagattgtaatgtttttacatatctgtccatctttttcagcaatgacccactttttacagaagcaggcatggtgccagcataaattttcatatgct

SEQ ID NO: 2 - protein, 1161 aa, Homo sapiens
TFGTVLLLSVLASYHGFNLDVEEPTIFQEDAGGFGQSVVQFGGSRLVVGAPLEVVAANQTGRLYDCAAATGMCQPIPLHIRPEAVNMSLGLTLAASTNGSRLLACGPTLHRVCGENSYSKGSCLLLGSRWEIIQTVPDATPECPHQEMDIVFLIDGSGSIDQNDFNQMKGFVQAVMGQFEGTDTLFALMQYSNLLKIHFTFTQFRTSPSQQSLVDPIVQLKGLTFTATGILTVVTQLFHHKNGARKSAKKILIVITDGQKYKDPLEYSDVIPQAEKAGIIRYAIGVGHAFQGPTARQELNTISSAPPQDHVFKVDNFAALGSIQKQLQEKIYAVEGTQSRASSSFQHEMSQEGFSTALTMDGLFLGAVGSFSWSGGAFLYPPNMSPTFINMSQENVDMRDSYLGYSTELALWKGVQNLVLGAPRYQHTGKAVIFTQVSRQWRKKAEVTGTQIGSYFGASLCSVDVDSDGSTDLILIGAPHYYEQTRGGQVSVCPLPRGQRVQWQCDAVLRGEQGHPWGRFGAALTVLGDVNEDKLIDVAIGAPGEQENRGAVYLFHGASESGISPSHSQRIASSQLSPRLQYFGQALSGGQDLTQDGLMDLAVGARGQVLLLRSLPVLKVGVAMRFSPVEVAKAVYRCWEEKPSALEAGDATVCLTIQKSSLDQLGDIQSSVRFDLALDPGRLTSRAIFNETKNPTLTRRKTLGLGIHCETLKLLLPDCVEDVVSPIILHLNFSLVREPIPSPQNLRPVLAVGSQDLFTASLPFEKNCGQDGLCEGDLGVTLSFSGLQTLTVGSSLELNVIVTVWNAGEDSYGTVVSLYYPAGLSHRRVSGAQKQPHQSALRLACETVPTEDEGLRSSRCSVNHPIFHEGSNGTFIVTFDVSYKATLGDRMLMRASASSENNKASSSKATFQLELPVKYAVYTMISRQEESTKYFNFATSDEKKMKEAEHRYRVNNLSQRDLAISINFWVPVLLNGVAVWDVVMEAPSQSLPCVSERKPPQHSDFLTQISRSPMLDCSIADCLQFRCDVPSFSVQEELDFTLKGNLSFGWVRETLQKKVLVVSVAEITFDTSVYSQLPGQEAFMRAQMEMVLEEDEVYNAIPIIMGSSVGALLLLALITATLYKLGFFKRHYKEMLEDKPEDTATFSGDDFSCVAPNVPLS